(19) United States
(12) Patent Application Publication
(10) Pub. No.: US 2026/0229305 A1
(43) Pub. Date: Aug. 6, 2026

(54) METABOLIC NETWORK PROCESSING SYSTEM AND METABOLIC NETWORK PROCESSING METHOD

(71) Applicants: KYOTO UNIVERSITY, Kyoto-shi, Kyoto (JP); Hitachi, Ltd., Tokyo (JP)

(72) Inventors: Miwa SATO, Tokyo (JP); Maiko TANABE, Tokyo (JP); Koichi WATANABE, Tokyo (JP); Atsushi MOCHIZUKI, Kyoto (JP); Takashi OKADA, Kyoto (JP)

(21) Appl. No.: 19/152,357

(22) PCT Filed: Apr. 18, 2023
(86) PCT No.: PCT/JP2023/015494

§ 371 Date: Jul. 30, 2025

(51) Int. Cl. G16B 5/00

(52) U.S. Cl. CPC G16B 5/00

(57) ABSTRACT

The present disclosure proposes a metabolic network processing system for processing a metabolic network corresponding to input host information in order to accurately extract pathways effective in substance production in consideration of an influence of modification in a metabolic network. The metabolic network processing system includes a storage device for holding a metabolic network processing program, and a control device for processing the metabolic network according to the program. The control device executes processing of estimating, by executing (a) a flux balance analysis calculation, (b) a structure sensitivity analysis calculation, or (c) a flux balance analysis calculation and an elementary mode analysis on the metabolic network including a starting substance and a target substance, a utilization pathway used for producing the target substance from the starting substance, processing of identifying a buffer structure in the utilization pathway of the metabolic network by executing a buffer structure calculation using the estimated utilization pathway, and processing of outputting information on the utilization pathway and information on the buffer structure (see FIG. 4).

### TECHNICAL FIELD

The present disclosure relates to a metabolic network processing system and a metabolic network processing method.

### BACKGROUND ART

In substance synthesis by an organism, a target substance is produced using the organism by artificially changing metabolism performed by the organism. The metabolism of an organism can be expressed by a network structure including a large number of biological reactions (metabolic network). Many biological reactions are carried out by genes, and therefore, many artificial modifications are made by adding or deleting genes to or from the metabolic network. Then, an effect of gene addition/deletion to/from the metabolic network is calculated.

For example, PTL 1 discloses a method for determining a metabolic flux that affects substance production. PTL 2 discloses a method for determining all metabolic fluxes from minimum analytical: information obtained by sample analysis of microorganisms and media during culture.

NPL 1 discloses that a flux distribution on a metabolic network is estimated by performing an operation by linear programming from a stoichiometric matrix using flux balance analysis (FBA) which is a method for analyzing a metabolic flux of an organism. This is a method for calculating an effect of addition or deletion of a gene as a flux distribution, and is a technique of estimating a gene for improving production of a target substance from a metabolic flux distribution which is a calculation result by attempting addition or deletion of a gene many times with a specific condition as a constraint term. NPL 2 discloses that a basis vector of a stoichiometric matrix is calculated according to elementary mode analysis (EMA), metabolism is divided into minimum reaction sets satisfying a steady state, and a linear sum of the reaction sets is used as a metabolic flux distribution.

On the other hand, unlike NPL 1 and NPL 2, NPL 3 discloses that an influence of perturbation is determined as a change in concentration of a substance on a metabolic network according to a method (structure sensitivity analysis) of calculating the influence of perturbation in the metabolic network. NPLs 4 and 5 disclose that a localization rule has been found by structure sensitivity analysis, such that the influence of perturbation is limited to a specific range of the network.

### CITATION LIST

### Patent Literature

PTL 1: JP2005-58226A

PTL 2: JP2005-293394A

### Non Patent Literature

NPL 1: D. Mccloskey, B. O. Palsson & A. M. Feist: Mol. Syst. Biol., 9, 661(2013 )

NPL 2: C. T. Trinh, A. Wlaschin & F. Srienc: Appl. Microbiol. Biotechnol., 81, 813(2009 )

NPL 3: A. Mochizuki and B. Fiedler, J. Theor. Biol. 367, 189(2015 )

NPL 4: A. Mochizuki and B. Fiedler, Math. Meth. Appl. Sci. 38, 3519(2015 ). 17

NPL 5: T. Okada and A. Mochizuki, Phys. Rev. Lett. 117, 048101(2016 )

### SUMMARY OF INVENTION

### Technical Problem

However, in the methods of using FBA according to PTLs 1 and 2 and NPLs 1 and 2, the target metabolism for which the metabolic flux distribution is calculated is freely (not theoretically but freely and empirically) selected, and the range of the influence of modification (addition and deletion of genes) is not considered. Therefore, it is not possible to predict what kind of change occurs in metabolism due to modification. Although it is possible to simulate the effects of deletion and addition of genes and changes in conditions according to the techniques disclosed in these literatures, it is not a technique capable of directly proposing a gene effective in substance production.

Further, according to PTL 1 and PTL 2, a pathway effective in substance production is being searched for. However, in PTL 1 and PTL 2, the pathway search includes arbitrariness and randomness, and it is not possible to accurately search for a pathway effective in substance production.

In view of such circumstances, the present disclosure provides a technique of accurately extracting a pathway effective in substance production in consideration of the influence of modification on a metabolic network.

### Solution to Problem

In order to solve the above problems, the present disclosure proposes, as an example, a metabolic network processing system for processing a metabolic network corresponding to input host information. The metabolic network processing system includes: a storage device configured to hold a program for processing the metabolic network; and a control device configured to read the program from the storage device and process the metabolic network. The control device is configured to execute processing of estimating, by executing (a) a flux balance analysis calculation, (b) a structure sensitivity analysis calculation, or (c) a flux balance analysis calculation and an elementary mode analysis on the metabolic network including a starting substance and a target substance, a utilization pathway used for producing the target substance from the starting substance, processing of identifying a buffer structure in the utilization pathway of the metabolic network by executing a buffer structure calculation using the estimated utilization pathway, and processing of outputting information on the utilization pathway and information on the buffer structure.

Additional features related to the present disclosure will be clarified from the description of the present description and the accompanying drawings. Aspects of the present disclosure may be achieved and implemented using elements, combinations of various elements, the following detailed description, and accompanying claims.

The description of the present description is merely a typical illustration and does not limit the scope of the claims or application examples of the present disclosure in any sense.

### Advantageous Effects of Invention

According to the technique of the present disclosure, it is possible to accurately extract a pathway effective in substance production in consideration of the influence of modification on a metabolic network.

### BRIEF DESCRIPTION OF DRAWINGS

FIG. 1 is a diagram illustrating an outline of a hardware structure of a metabolic network processing system 10 according to the present embodiment.

FIG. 2 is a diagram illustrating an outline of a software configuration of the metabolic network processing system 10 according to the present embodiment.

FIG. 3 is a flowchart illustrating metabolic network processing according to the present embodiment.

FIG. 4 is a diagram illustrating an example of a pathway configuration of a metabolic network in each step (from step 1 to step 4) of the metabolic network processing.

FIG. 5A is a diagram illustrating an example of a GUI when metabolic network data is acquired from a metabolic information DB (for example, a public DB) and the acquired metabolic network data.

FIG. 5B is a diagram illustrating an example of a GUI when metabolic network data related to a starting substance and a target (final) substance is extracted from the acquired metabolic network ((complicated metabolic network) public DB) and the extracted metabolic network data.

FIG. 5C is a diagram illustrating an example of a GUI when a utilization pathway is estimated from the extracted metabolic network data and metabolic network data in which the utilization pathway is illustrated.

FIG. 5D is a diagram illustrating an example of a GUI when an influence pathway affecting an amount of substance is estimated from the metabolic network data in which the utilization pathway is estimated and metabolic network data in which the influence pathway is illustrated.

### DESCRIPTION OF EMBODIMENTS

The present embodiment proposes a metabolic design method characterized in that underlying technologies (structure sensitivity analysis and buffer structure calculation) are connected and logically implemented as a technology that can be seamlessly implemented without randomness due to many trials or human intervention due to free selection. Hereinafter, the present embodiment will be described with reference to the accompanying drawings.

In the present description, the term “enzyme” refers to a biomolecule having a function of converting (catalyzing) a certain substance (compound) into another substance. The term “metabolism” refers to a process of conversion into a substance or energy by a series of enzyme reactions. The term “metabolic network” refers to a network in which a substance generated with another enzyme is changed to another substance with another enzyme, and the transition is expressed as a network. The term “flux (Flux=flow velocity)” refers to a flow of transition of a substance, or a speed or an amount thereof. The flux analysis refers to analysis of the flow, speed, and amount of transition of a substance. The term “flux balance analysis (FBA)” refers to a technique of expressing a flux of a metabolic network by a matrix (stoichiometric matrix) and simulating a transition of conversion of a substance that cannot be visually observed. The term “structure sensitivity analysis (SSA)” is a name of a unique method in which a “structure” is uniquely added to a general word representing the type of analysis called “sensitivity analysis”, and refers to a technique of proposing an enzyme that affects an amount of a substance on a metabolic network from the structure (form) of the metabolic network. The term “localization rule” refers to a theorem that defines which range of a metabolic network is controlled by each enzyme in terms of a network structure. The term “buffer structure” refers to a subnetwork divided in consideration of the range of influence according to the localization rule. The term “elementary mode analysis (EMA)” refers to a theory of dividing a metabolic network into subnetworks. When the subnetworks are superimposed, the original network is obtained, but the range of influence is not considered. The term “elementary flux mode (EFM)” refers to a subnetwork calculated by the EMA.

### Example of Hardware Structure of Metabolic Network Processing System

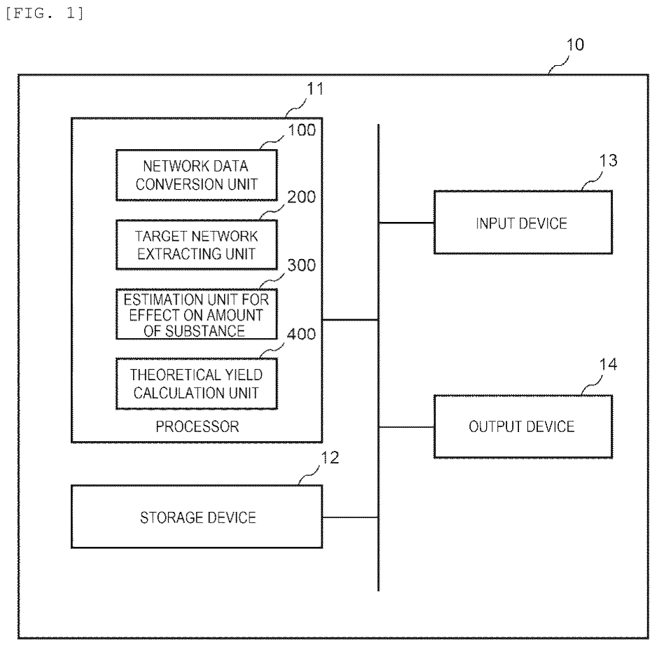

FIG. 1 is a diagram illustrating an outline of a hardware structure of a metabolic network processing system 10 according to the present embodiment. The metabolic network processing system 10 can be implemented by a computer system, and includes, for example, a control device 11, a storage device 12, an input device 13, and an output device 14.

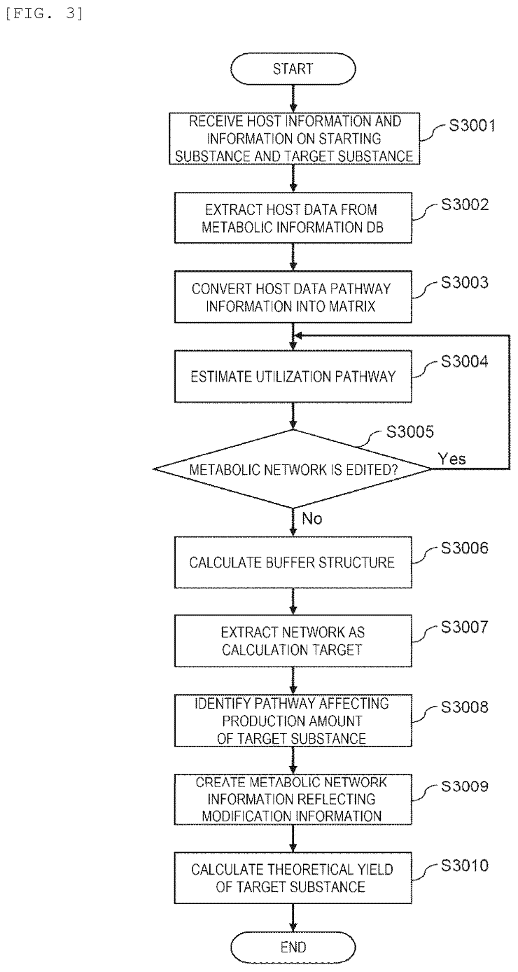

The control device 11 reads a metabolic network processing program (see FIG. 3) including processing of extracting a metabolic network in consideration of a range of influence of genetic modification from the storage device 12, loads the program in an internal memory (not illustrated), and implements a network data conversion unit 100, a target network extracting unit 200, an estimation unit 300 for effect on an amount of substance, and a theoretical yield calculation unit 400.

The storage device 12 holds the metabolic network processing program that executes metabolic network extraction processing in consideration of the range of influence of genetic modification, processing of determining a metabolic flux under designated conditions (for example, culture conditions), and processing of identifying and outputting a gene or enzyme as modification target that is effective in production of a target substance in the metabolic flux. The metabolic network processing program is a program corresponding to the flowchart in FIG. 4 to be described below.

The input device 13 is implemented by, for example, a keyboard, a mouse, and a switch. The input device 13 can be used when an operator (user) inputs host information, information on a starting substance and a target substance, various instructions, and the like to the metabolic network processing system 10.

The output device 14 is implemented by, for example, a display device and a printer. The output device 14 displays, for example, a result of processing the metabolic network (buffer structure information, utilization pathway information, modification target gene list, theoretical yield information, and the like, which will be described below) on a display screen or prints the result on a printing medium.

In FIG. 1, as the computer system, the control device 11, the storage device 12, the input device 13, and the output device 14 are connected via a data line/control line, but the metabolic network processing system 10 may be configured such that the devices are remotely disposed and connected via a communication network.

### Example of Software Configuration of Metabolic Network Processing System

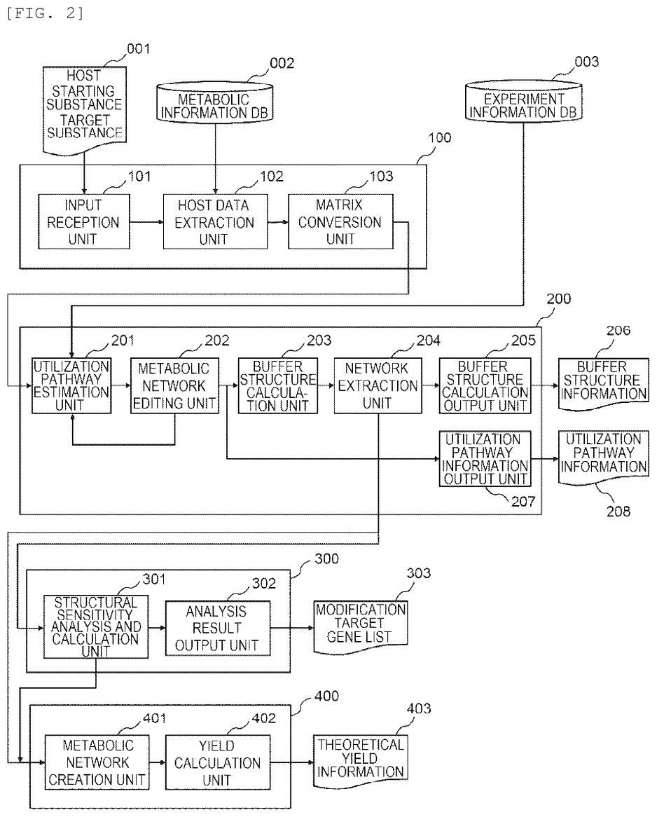

FIG. 2 is a diagram illustrating an outline of a software configuration of the metabolic network processing system 10 according to the present embodiment. FIG. 2 mainly illustrates an example of internal configurations of the network data conversion unit 100, the target network extracting unit 200, the estimation unit 300 for effect on amount of substance, and the theoretical yield calculation unit 400.

### (i) Network Data Conversion Unit 100

The network data conversion unit 100 includes an input reception unit 101, a host data extraction unit 102, and a matrix conversion unit 103 as an internal configuration.

The input reception unit 101 receives, as input information 001, information on a host as an analysis target, information on a target starting substance, and information on the target substance as a target (host information/starting substance/target substance).

The host data extraction unit 102 acquires metabolic network data on the host received by the input reception unit 101 from a metabolic information DB 002 (a DB held in the storage device 12 or a DB provided externally) storing an organism metabolic network. The host data extraction unit 102 extracts a pathway containing the received starting substance and target substance from the acquired metabolic network data on the host. Note that a public database such as KEGG or Metacyc can be used as the metabolic information DB 002. However, the metabolic information DB 002 may be configured using or added to unique held experiment data called in-house data held by each company.

The matrix conversion unit 103 converts pathway information on the metabolic network extracted by the host data extraction unit 102 into matrix data. At this time, it is preferable to acquire a large amount of information so as to cover pathways including the starting substance and the target substance as much as possible.

### (ii) Target Network Extracting Unit 200

The target network extracting unit 200 includes a utilization pathway estimation unit 201, a metabolic network editing unit 202, a buffer structure calculation unit 203, a network extraction unit 204, a buffer structure calculation output unit 205, and a utilization pathway information output unit 207. The target network extracting unit 200 performs processing of extracting, from the matrix data, a network having a minimum configuration, which includes a starting substance and a target substance.

The utilization pathway estimation unit 201 estimates a utilization pathway and an unnecessary pathway by performing, for the matrix data, a calculation based on flux balance analysis (FBA), the structure sensitivity analysis in which experimental information is acquired from an experimental information DB 003 and substance concentration information included in the experimental information is used, or a calculation based on FBA +elementary mode analysis (EMA). The utilization pathway and the reversibility and irreversibility of the enzyme vary depending on conditions such as culture conditions and mutation conditions. Therefore, an object of the processing is to determine a metabolic pathway under a specific condition. At this time, a pathway may be deleted or added using experimental information such as a substance concentration.

The metabolic network editing unit 202 performs processing of adding or deleting a pathway in the metabolic network according to an instruction from an operator or an automatic editing program for random selection. When a structure of the metabolic network is changed, the processing may return to the utilization pathway estimation unit 201 to recalculate the utilization pathway.

The buffer structure calculation unit 203 performs the buffer structure calculation using the utilization pathway under a determined designated condition.

The network extraction unit 204 extracts a network as a calculation target based on a buffer structure calculation result obtained by the buffer structure calculation unit 203.

The buffer structure calculation output unit 205 outputs buffer structure information 206 represented in a list format. The buffer structure information 206 can be, for example, stored in a buffer structure information DB (not illustrated), displayed on a display screen, or output by a printer.

The utilization pathway information output unit 207 outputs, as utilization pathway information 208 (file), the utilization pathway estimated (identified) by the utilization pathway estimation unit 201 and the metabolic network editing unit 202. The utilization pathway information 208 can be stored in a utilization pathway information DB (not illustrated), displayed on a display screen, or output by a printer. The operator can use the output utilization pathway information 208 for interpretation and consideration of a processing result.

(iii) Estimation Unit 300 for Effect on Amount of Substance

The estimation unit 300 for effect on amount of substance includes a structure sensitivity analysis calculation unit 301 and an analysis result output unit 302.

The structure sensitivity analysis calculation unit 301 performs a calculation based on the structure sensitivity analysis using, as an input, the metabolic network extracted by the network extraction unit 204, and identifies a pathway that affects a production amount of the target substance.

The analysis result output unit 302 outputs, as a modification target gene list 303, a list of genes and/or enzymes on a pathway as a promotion or reduction target. The modification target gene list 303 can be, for example, stored in a modification target gene list DB (not illustrated), displayed on a display screen, or output by a printer.

### (iv) Theoretical Yield Calculation Unit 400

The theoretical yield calculation unit 400 includes a metabolic network creation unit 401 and a yield calculation unit 402.

The metabolic network creation unit 401 receives the calculation result 4 the structure sensitivity analysis calculation unit 301 and the metabolic network information extracted by the target network extracting unit 200, and creates metabolic network information reflecting modification information.

The yield calculation unit 402 receives the metabolic network information after the modification, calculates a theoretical yield of the target substance with respect to the amount of substance of the starting substance according to the flux balance analysis (FBA), and outputs the theoretical yield as theoretical yield information 403. The theoretical yield information 403 can be, for example, stored in a theoretical yield information DB (not illustrated), displayed on a display screen, or output by a printer.

### Details of Metabolic Network Processing

FIG. 3 is a flowchart illustrating metabolic network processing according to the present embodiment. In the following description of each step, an operation subject is each processing unit (the input reception unit 101, the host data extraction unit 102, . . . , and the like) illustrated in FIG. 2. However, each processing unit is implemented by loading a program in the control device 11, and therefore, an operation subject of each step may be replaced with the control device 11. FIG. 4 is a diagram illustrating an example of a pathway configuration of a metabolic network in each step (from step 1 to step 4) of the metabolic network processing.

### (i) S3001

The input reception unit 101 receives, as the input information 001, information on a host, information on a starting substance, and information on a target substance, which are analysis targets. The input information 001 is, for example, information input by the operator via the input device 13, and includes information on the host (for example, a name of species of an organism) and the information on the starting substance and the information on the target substance in a metabolic network. Here, the host refers to an organism used for substance production, and examples thereof include Escherichia coli, actinomycetes, mold, yeast, bacteria, and plants. These organisms may be not only in a natural state but also organisms into which genes of other organisms are incorporated.

### (ii) S3002

The host data extraction unit acquires metabolic network data on the host from the metabolic information DB 002 based on the received information on the host (the name of the species of the organism). Since there is a one-to-one correspondence between the metabolic network and the information on the host, the metabolic network data on the host can be uniquely acquired. As illustrated in step 1 of FIG. 4, the metabolic network data acquired herein is data constituted by a complicated network including as many pieces of information on substances and genes other than the starting substance and the target substance as possible.

(iii) S3003

The matrix conversion unit 103 converts the metabolic network data (complicated network) acquired in S3002 into matrix data so that the data can be operated in subsequent processing. A large number of reaction formulas exist in the metabolic network, and these reaction formulas are listed and expressed as a matrix (see NPL 3 to NPL 5).

### (iv) S3004

The utilization pathway estimation unit 201 estimates a utilization pathway and an unnecessary pathway in the metabolic network data (complicate network) (step 2 in FIG. 4: network extracted according to localization rule) by (a) executing flux balance analysis (FBA) operation (see NPL 1 and NPL 2 for details of FBA), (b) acquiring substance concentration information (information on the concentration of a starting substance, information on the concentration of a target substance, or information on the concentration of other substances) from experimental information DB 003 and executing structure sensitivity analysis (see NPL 3 and NPL 4 for details of structure sensitivity analysis), or (c) executing combination operation of FBA and elementary mode analysis (EMA) (see NPL 1 and NPL 2 for details of FBA, and PTL 2 for EMA) on matrix data of the metabolic network obtained in S3003. Although the arbitrariness and randomness of the metabolic network are not eliminated in the utilization pathway estimation using (a) FBA and (c) FBA +EMA, the arbitrariness and randomness of the metabolic network are eliminated in the utilization pathway estimation using (b) experimental information+structure sensitivity analysis. When the utilization pathway estimation using (a) FBA or (c) FBA+EMA is performed, arbitrariness and randomness are eliminated by performing the buffer structure calculation to be described below.

### (v) S3005

The metabolic network editing unit 202 determines whether editing (substance concentration change, deletion/addition of pathway, or the like) has been performed on the metabolic network whose utilization pathway has been estimated in S3004. The presence or absence of editing can be determined by whether an instruction to change the substance concentration or the like is input by the operator or whether a program for automatically performing editing is operating.

If the metabolic network is being edited (Yes in S3005), the processing returns to S3004. In this case, the utilization pathway estimation unit 201 performs the operations of (a) to (c) according to the changed condition and the changed pathway, and repeats the editing (S3005) and the utilization pathway estimation (S3004) until the experimental information input in an auxiliary manner matches the operation result.

On the other hand, when the metabolic network is not edited (No in S3005: including a case where new editing is not performed), the processing proceeds to S3006.

The utilization pathway in the metabolic network is determined by the processing of S3005.

### (vi) S3006

The buffer structure calculation unit 203 performs a buffer structure analysis using the utilization pathway determined in S3005, and divides the metabolic network into a plurality of ranges. Details of the buffer structure analysis are described in NPL 5.

The buffer structure analysis is to calculate a range in which the influence of metabolism remains (closed). For example, a range indicated by a dotted line or a dash-dotted line in step 2 of FIG. 4 is the buffer structure. The difference in the type of line indicates the difference in the range of influence. In this way, when the buffer structure is calculated, for example, the range of each nested structure is identified.

(vii) S3007

The network extraction unit 204 extracts a metabolic network as a calculation target from the buffer structure identified in S3006. The metabolic network to be extracted is a buffer structure including a starting substance (input) and a target substance (output), and is uniquely determined based on the buffer structure analyzed by S3006. That is, all buffer structures between the starting substance and the target substance (for example, the buffer structure indicated by the dotted line and the buffer structure indicated by the dash-dotted line in step 2 of FIG. 4) are extracted.

In parallel with the extraction of the metabolic network by the network extraction unit 204, the buffer structure calculation output unit 205 Outputs the buffer structure information 206 represented in, for example, a list format (for example, the same format as the data held in the database (DB)). The buffer structure information 206 can be, for example, stored in a buffer structure information DB (not illustrated), displayed on a display screen, or output by a printer.

Further, in parallel, the utilization pathway information output unit 207 outputs, as the utilization pathway information 208 (file), the utilization pathway estimated (identified) by the utilization pathway estimation unit 201 and the metabolic network editing unit 202. Note that not all of the extracted parts of the metabolic network are utilization pathways. Among the networks illustrated in step 2 of FIG. 4, for example, when pathways inside the buffer structure indicated by the dotted line are not used to generate the target substance from the starting substance, the pathway excluding this part is output as the utilization pathway information 208 (step 3 of FIG. 4). The utilization pathway information 208 can be stored in a utilization pathway information DB (not illustrated), displayed on a display screen, or output by a printer. The operator can use the output utilization pathway information 208 for interpretation and consideration of the processing result.

(viii) S3008

The structure sensitivity analysis calculation unit 301 receives the metabolic network extracted in S3007 by the network extraction unit 204, performs the structure sensitivity analysis, and identifies a pathway that affects the production amount of the target substance. That is, the structure sensitivity analysis calculation unit 301 estimates which flow of paths on the metabolic network extracted to improve the production amount of the target substance needs to be strengthened.

When the structure sensitivity analysis calculation is executed, matrix data (listed in descending order of numerical values (scores) ) indicating the influence of strengthening of a specific path on the extracted metabolic network on each substance by “+” and “−” is output as a calculation result. Therefore, an efficient method for enhancing the target substance production amount can be determined by observing the matrix.

The analysis result output unit 302 outputs the matrix data (a list of genes and/or enzymes on a pathway to be promoted or decreased) as the modification target gene list 303. The modification target gene list 303 can be, for example, stored in a modification target gene list DB (not illustrated), displayed on a display screen, or output by a printer.

### (ix) S3009

The metabolic network creation unit 401 receives the metabolic network extracted in S3007 and the structure sensitivity analysis result (matrix data) calculated in S3008, and creates metabolic network information reflecting modification information. More specifically, the metabolic network creation unit 401 creates the metabolic network information reflecting the modification information by considering (reflecting) the analysis result (which gene and which path need to be enhanced and which path needs be weakened) obtained by the structure sensitivity analysis calculation in S3008 in the metabolic network extracted in S3007 (step 4 in FIG. 4). For example, as illustrated in step 4 of FIG. 4, among the utilization pathways identified (estimated) in step 3, a pathway indicated by a thick line is identified as a pathway (substance amount affecting pathway) for improving the production amount of succinic acid.

### (x) S3010

The yield calculation unit 402 calculates, based on the metabolic network information after the modification, a theoretical yield of the target substance with respect to the amount of substance of the starting substance according to the flux balance analysis (FBA), and outputs the theoretical yield as theoretical yield information 403. According to FBA, for example, a calculation amount of succinic acid with respect to an amount of saccharides (for example, 100 g) in the metabolic network after modification is calculated.

The theoretical yield information 403 can be, for example, stored in a theoretical yield information DB (not Our illustrated), displayed on a display screen, or output by a printer.

### GUI and Screen Transition Diagram

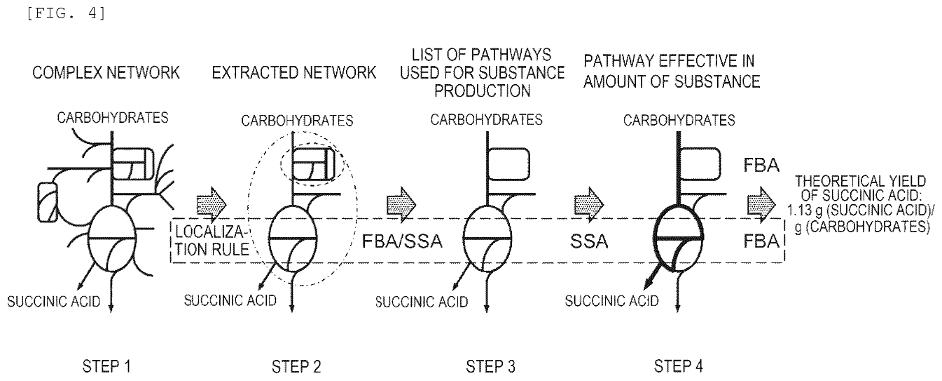

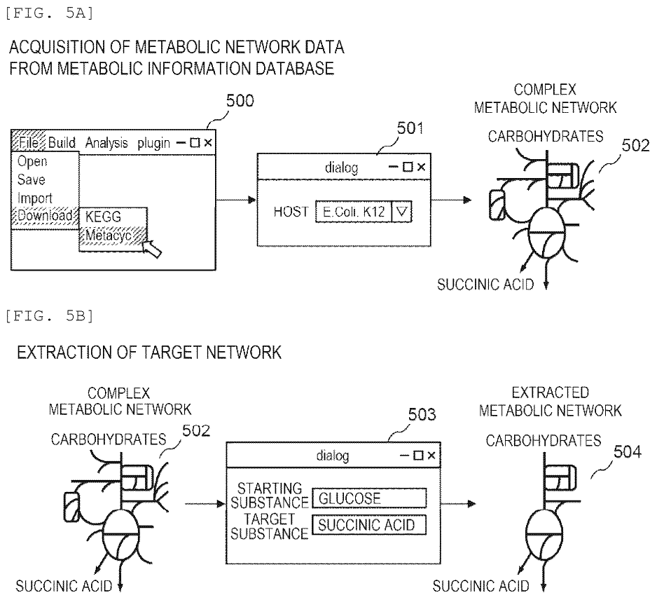

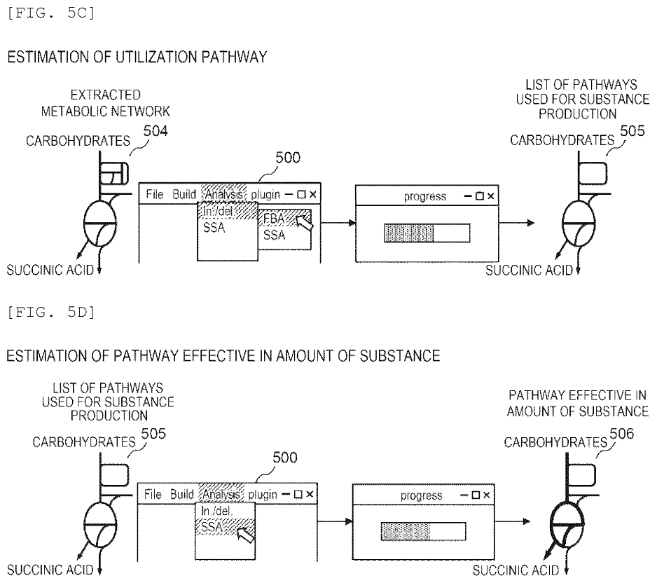

FIGS. 5A to 5D are diagrams illustrating examples of a graphical user interface (GUI) operated by the operator, and transition of a metabolic network displayed on a screen by a GUI operation in the metabolic network processing system 10 of the present embodiment. FIG. 5A is a diagram illustrating an example of a GUI when metabolic network data is acquired from a metabolic information DB (for example, a public DB) and the acquired metabolic network data. FIG. 5B is a diagram illustrating an example of a GUI when metabolic network data related to a starting substance and a target (final) substance is extracted from the acquired metabolic network ((complicated metabolic public DB) and the extracted metabolic network data. FIG. 5C is a diagram illustrating an example of a GUI when a utilization pathway estimated from the extracted metabolic network data and metabolic network data in which the utilization pathway is illustrated. FIG. 5D is a diagram illustrating an example of a GUI when an influence pathway affecting an amount of substance is estimated from the metabolic network data in which the utilization pathway is estimated and metabolic network data in which the influence pathway is illustrated.

As shown in FIG. 5A, when the operator selects File→Download→Metacyc in a GUI500, a host information (host name) input dialog 501 and a start/target substance input dialog 503 (see FIG. 5B) are displayed. When the operator inputs a host name to the host information input dialog 501 and executes a search (the input reception unit 101 receives the host information and the like), the host data extraction unit 102 acquires target metabolic network data from the metabolic information DB 002 and displays the data on the display screen. Here, the acquired metabolic network corresponds to the host name and is a complicated network including a network in a range having low relevance to the starting substance and the target substance. The start/target substance input dialog 503 may be displayed simultaneously with the host information input dialog 501, or may be displayed after the metabolic network corresponding to the host information is acquired.

Next, as illustrated in FIG. 5B, the utilization pathway estimation unit 201 extracts the metabolic network as a calculation target from the complicated metabolic network 502 (the extracted metabolic network 504) based on the substance information input to the start/target substance input dialog 503 and the localization rule.

Subsequently, as illustrated in FIG. 5C, when the operator inputs an instruction via the GUI 500 to execute an FBA calculation or the like (FBA, SSA, or FBA+EMA) on the extracted metabolic network 504, the network extraction unit 204 executes a designated operation and identifies (estimates) a utilization pathway (estimated utilization pathway 505).

Finally, as illustrated in FIG. 5D, when the operator inputs an instruction via the GUI 500 to execute SSA on the estimated utilization pathway 505, the structure sensitivity analysis calculation unit 301 executes the structure sensitivity analysis on the estimated utilization pathway 505 and identifies a pathway that affects the production amount of the target substance from the starting substance (a pathway 506 effective in the amount of substance).

### Summary

In the present embodiment, the structure sensitivity analysis and the buffer structure calculation are introduced into common metabolic network modification processing in which there is no pipeline capable of performing a series of calculations and randomness and arbitrariness cannot be eliminated. Thereby, randomness (modifications based on the results of multiple trial and error) and arbitrariness (modifications based on an experience point) network modification are eliminated. The structure sensitivity analysis and the buffer structure calculation themselves are known techniques, and the present embodiment is characterized in that these are applied to the flux balance analysis calculation (FBA).

(i) According to the present embodiment, the control device 11 estimates, by executing (a) a flux balance analysis calculation (FBA), (b) a structure sensitivity analysis calculation, or (c) the flux balance analysis calculation (FBA) and an elementary mode analysis (EMA) on the metabolic network containing a starting substance and a target substance, a utilization pathway used for producing the target substance from the starting substance. Then, the control device 11 identifies a buffer structure in the utilization pathway of the metabolic network by executing a buffer Structure calculation using the estimated utilization pathway, and outputs information on the utilization pathway and information on the buffer structure. In this way, it is possible to utilize information (the buffer structure information and the utilization pathway information) important for designing the metabolic network (modifying genes and enzymes) in order to improve the production of the target substance. That is, it is possible to eliminate randomness (modifications based on the results of multiple trial and error) and arbitrariness (modification based on an experience point) when modifying the metabolic network.

Specifically, in the case of the above (b), the control device 11 executes the structure sensitivity analysis calculation, using the experimental measurement value indicating a substance concentration, to estimate the utilization pathway. The experimental measurement value (actual experimental data) is used, and therefore, it is possible to logically estimate the utilization pathway while eliminating arbitrariness at this stage (before calculating the buffer structure). In the metabolic network processing system 10, it is possible to edit (add or delete a pathway, or the like) the target metabolic network even when the utilization pathway is once estimated. In this case, when an instruction to edit the pathway of the metabolic network for which the utilization pathway has been estimated is input (when an instruction to edit is input by the operator, or when an instruction to edit is Our input by a program set to edit randomly), the control device 11 estimates the utilization pathway again on the metabolic network for which editing has been performed. In this way, it is possible to estimate an optimal utilization pathway in a desired metabolic network.

(ii) The control device 11 extracts, based on the estimated buffer structure in the utilization pathway, a metabolic network including only the buffer structure required to produce the target substance from the starting substance (extracted metabolic network). As described above, the buffer structure refers to a structure in which the influence of metabolism remains. That is, the metabolic network involved in the production of target substances from starting substances is configured such that several buffer structures are nested. That is, when the starting substance and the target substance are determined and the buffer structure is clarified, only the buffer structure related to production of the target substance from the starting substance can be easily extracted.

Then, the control device 11 executes the structure sensitivity analysis calculation on the extracted metabolic network, and identifies a pathway that affects production of the target substance in the extracted metabolic network.

(iii) The control device 11 further outputs, as the modification target gene list 303, information on a pathway that affects the production of the target substance. The modification target gene list 303 is obtained by executing the structure sensitivity analysis calculation on the extracted metabolic network. The modification target gene list 303 indicates which path flow on the metabolic network needs to be enhanced in order to increase the production amount of the target substance, and is output as matrix data. More specifically, in the modification target gene list 303, what kind of influence is exerted on each substance when which path is enhanced can be indicated by “+” and “−”. By observing this matrix data, it is possible to know a method of enhancing the target substance production with high efficiency as a network. Alternatively, the modification target gene list 303 may be output by, for example, scoring the modification targets in descending order of influence on target substance production. In this case, a high score may be given to a modification target having a favorable influence, and at the same time, a score of a part deteriorating by the modification may be added.

(iv) The control device 11 further generates information on a metabolic network reflecting modification information, based on the information on the buffer structure and the information on the pathway affecting the production of the target substance (see FIG. 4: a network indicated by a thick line in step 4). Then, the control device 11 calculates a theoretical yield of the target substance with respect to an amount of the starting substance by executing the flux balance analysis calculation using the information on the metabolic network reflecting the modification information. Accordingly, the modification of the metabolic network can be quantitatively evaluated.

(v) Functions of the present embodiment can also be implemented by a software program code. In this case, a storage medium that records the program code is provided in a system or a device, and a computer (or CPU or MPU) of the system or the device reads the program code stored in the storage medium. In this case, the program code read from the storage medium implements the functions of the above-described embodiment, and the program code, per se, and the storage medium that stores the program code constitute the present disclosure. Examples of the storage medium for supplying such program codes include a flexible disk, a CD-ROM, a DVD-ROM, a hard disk, an optical disk, a magneto-optical disk, a CD-R, a magnetic tape, a nonvolatile memory card, and a ROM.

An operating system (OS) or the like operating on a computer may perform a part or all of actual processing based on an instruction of the program codes, and the functions of the embodiments described above may be implemented by the processing. Further, after the program codes read from the storage medium are written in a memory on the computer, a CPU or the like of the computer may perform a part or all of actual processing based on an instruction of the program codes, and the functions of the embodiments described above may be implemented by the processing.

The software program codes for implementing the functions of the embodiments and each example may be stored, by distributing via a network, in a storage unit such as a hard disk or a memory of the system or the device or a storage medium such as a CD-RW a CD-R, and the computer (or CPU or MPU) of the system or the device may read and execute the program codes stored in the storage unit or the storage medium at the time of use.

The process and technique described here are not essentially related to any specific device and can be implemented by a combination of each component. In addition, various types of general-purpose devices can be added. A dedicated device may be constructed to execute the functions of the present embodiment and each example. Various functions can be formed by appropriately combining a plurality of components in the present embodiment and each example. For example, some components may be deleted from all the components shown in the embodiment and each example, or components in different examples may be appropriately combined.

In the present disclosure, specific examples are described, but these are for description (understanding of the technology of the present disclosure) without limitation in all viewpoints. Those skilled in the art will readily recognize that there are numerous combinations of hardware, software, and firmware that are suitable for implementing the techniques of the present disclosure. For example, the above-described software can be implemented in a wide range of programs or script languages such as assembler, C/C++, perl, Shell, PHP, and Java (registered trademark).

Control lines and information lines considered to be necessary for description are shown in the above-described embodiments, and not all control lines and information lines in a product are necessarily shown. All configurations may be connected to one another.

Those skilled in the art can clarify other implementations of the present disclosure from consideration of the present embodiment and each example. The description and the specific examples are merely typical, and the scope and spirit of the technology of the present disclosure are indicated by the following claims.

### REFERENCE SIGNS LIST

10: metabolic network processing system
11: control device
12: storage device
13: input device
14: output device
001: input information
002: metabolic information DB
003: experimental information DB
100: network data conversion unit
101: input reception unit
102: host data extraction unit
103: matrix conversion unit
200: target network extracting unit
201: utilization pathway estimation unit
202: metabolic network editing unit
203: buffer structure calculation unit
204: network extraction unit
205: buffer structure calculation output unit
206: buffer structure information
207: utilization pathway information output unit
208: utilization pathway information
300: effect estimation unit for amount of substance
301: structure sensitivity analysis calculation unit
302: analysis result output unit
303: modification target gene list
400: theoretical yield calculation unit
401: metabolic network creation unit
402: yield calculation unit
403: theoretical yield information
500: GUI
501: host information input dialog
502: complicate metabolic network
503: start/target substance input dialog
504: extracted metabolic network
505: estimated utilization pathway
506: pathway effective in amount of substance

1. A metabolic network processing system for processing a metabolic network corresponding to input host information, the metabolic network processing system comprising:
a storage device configured to hold a program for processing the metabolic network; and
a control device configured to read the program from the storage device and process the metabolic network, wherein
the control device is configured to execute,
processing of estimating, by executing (a) a flux balance analysis calculation, (b) a structure sensitivity analysis calculation, or (c) a flux balance analysis calculation and an elementary mode analysis on the metabolic network including a starting substance and a target substance, a utilization pathway used for producing the target substance from the starting substance,
processing of identifying a buffer structure in the utilization pathway of the metabolic network by executing a buffer structure calculation using the estimated utilization pathway, and
processing of outputting information on the utilization pathway and information on the buffer structure.

2. The metabolic network processing system according to claim 1, wherein
the control device is configured to execute the structure sensitivity analysis calculation, using an experimental measurement value indicating a substance concentration, to estimate the utilization pathway.

3. The metabolic network processing system according to claim 1, wherein
when an instruction to edit a pathway of the metabolic network for which the utilization pathway has been estimated is input, the control device is configured to execute processing of estimating the utilization pathway again on the metabolic network for which the editing has been performed.

4. The metabolic network processing system according to claim 1, wherein
the control device is further configured to extract, based on the buffer structure in the utilization pathway, a metabolic network including only the buffer structure required to produce the target substance from the starting substance, and to generate the extracted metabolic network.

5. The metabolic network processing system according to claim 4, wherein
the control device is further configured to execute processing of identifying a pathway affecting production of the target substance in the extracted metabolic network by executing the structure sensitivity analysis calculation on the extracted metabolic network.

6. The metabolic network processing system according to claim 5, wherein
the control device is further configured to execute processing of outputting information on the pathway affecting the production of the target substance as a modification target gene list.

7. The metabolic network processing system according to claim 5, wherein
the control device is further configured to execute processing of generating information on a metabolic network reflecting modification information, based on the information on the buffer structure and the information on the pathway affecting the production of the target substance.

8. The metabolic network processing system according to claim 7, wherein
the control device is further configured to execute processing of calculating a theoretical yield of the target substance with respect to an amount of the starting substance by executing the flux balance analysis calculation using the information on the metabolic network reflecting the modification information.

9. A metabolic network processing method for processing a metabolic network corresponding to input host information, the metabolic network processing method comprising:
estimating, by executing (a) a flux balance analysis calculation, (b) a structure sensitivity analysis calculation, or (c) a flux balance analysis calculation and an elementary mode analysis on the metabolic network including a starting substance and a target substance, a utilization pathway used for producing the target substance from the starting substance, by a control device configured to read, from a storage device holding a program for processing the metabolic network, the program and to process the metabolic network;
identifying a buffer structure in i the utilization pathway of the metabolic network by executing a buffer structure calculation using the estimated utilization pathway, by the control device; and
outputting information on the utilization pathway and information on the buffer structure, by the control device.

10. The metabolic network processing method according to claim 9, further comprising:
extracting, based on the buffer structure in the utilization pathway, a metabolic network including only the buffer structure required to produce the target substance from the starting substance, and generating the extracted metabolic network, by the control device.

11. The metabolic network processing method according to claim 10, further comprising:
executing, by the control device, processing of identifying a pathway affecting production of the target substance in the extracted metabolic network by executing the structure sensitivity analysis calculation on the extracted metabolic network.

12. The metabolic network processing method according to claim 11, further comprising:
outputting, by the control device, information on the pathway affecting the production of the target substance as a modification target gene list.

13. The metabolic network processing method according to claim 11, further comprising:
generating, by the control device, information on a metabolic network reflecting modification information, based on the information on the buffer structure and the information on the pathway affecting the production of the target substance.

14. The metabolic network processing method according to claim 13, further comprising:
calculating, by the control device, a theoretical yield of the target substance with respect to an amount of the starting substance by executing the flux balance analysis calculation using the information on the metabolic network reflecting the modification information.
